(19) United States
(12) Patent Application Publication
(10) Pub. No.: US 2026/0151755 A1
(43) Pub. Date: Jun. 4, 2026

(54) ENHANCED CATALYST FOR CARBON DIOXIDE HYDROGENATION TO METHANOL

(71) Applicant: Petroliam Nasional Berhad (PETRONAS), Kuala Lumpur (MY)

(72) Inventors: Nor Hafizah BERAHIM, Kajang (MY); Akbar ABU SEMAN, Kajang (MY); Nor Hafizah YASIN, Kajang (MY); Noor Asmawati MOHD ZABIDI, Kajang (MY)

(21) Appl. No.: 19/126,242

(22) PCT Filed: Nov. 1, 2023
(86) PCT No.: PCT/MY2023/050089

§ 371 Date: Apr. 30, 2025

(30) Foreign Application Priority Data
Nov. 1, 2022 (MY) PI2022006136

(51) Int. Cl. B01J 23/889; B01J 21/04; C07C 29/154; C07C 31/04

(52) U.S. Cl. CPC B01J 23/8892; B01J 21/04; C07C 29/154; C07C 31/04

(57) ABSTRACT

An enhanced catalyst for carbon dioxide hydrogenation to methanol includes a copper-zinc oxide catalyst supported on an alumina support and at least one promoter selected from manganese, niobium and zirconium. The catalyst is activated prior to being utilised in the process for carbon dioxide hydrogenation to methanol. A process for producing methanol by reacting carbon dioxide with hydrogen over the enhanced catalyst is also included.

### FIELD OF INVENTION

The invention relates to a catalyst, in particular for use in hydrogenation of carbon dioxide to methanol.

### BACKGROUND

Traditionally, methanol is commercially produced by reacting carbon monoxide and hydrogen over a catalyst, typically a mixture of copper and zinc oxides supported by alumina.

CO+2H₂→CH₃OH

An alternative method is to use carbon dioxide instead, using Cu/ZnO/silica as a catalyst, which has the added benefit of utilising carbon emissions and increasing carbon offset efforts.

CO₂+3H₂→CH₃OH+H₂O

However, the alternative method described above disadvantageously results in a low yield of less than 1%. While there have been attempts to improve the yield, such as with the use of promoted Cu/Zn catalyst supported by silica (SBA-15) which may increase the yield to approximately 19%, SBA-15 is not recyclable which results in very poor catalytic performance and is costly to implement.

An aim of the invention therefore is to provide a catalyst suitable for use in hydrogenation of carbon dioxide to methanol which overcomes at least some of the above issues.

### SUMMARY OF INVENTION

In an aspect of the invention, there is provided an enhanced catalyst for carbon dioxide hydrogenation to methanol comprising;
a copper-zinc oxide catalyst supported on an alumina support; and
at least one promoter;
characterized in that the promoter is added to the copper-zinc oxide catalyst in an amount of up to 1 wt. % to form a promoted copper-zinc oxide catalyst on the alumina support.

In one embodiment the promoted copper-zinc oxide catalyst is activated under a hydrogen flow within the range of 20 to 1000 ml/min, at a pressure within the range of 1 to 10 bar, at a reduction temperature within the range of 190 to 390° C., at a ramping rate within the range of 1 to 38° C./min for a duration of 1 to 7 hours.

Preferably the alumina support is aluminium oxide.

Advantageously the aluminium oxide support enables an outstanding catalytic performance with high CO₂ conversion, methanol selectivity and methanol yield. Additionally aluminium oxide in comparison to the support utilised in the prior art is more stable and cost efficient.

In one embodiment the at least one promoter is selected from manganese, niobium and zirconium.

Preferably the at least one promoter is a combination of manganese, niobium and zirconium, typically in the ratio of 4:1:1.

In one embodiment manganese is present in an amount of 0.06 wt. %, niobium is present in an amount of 0.015 wt. % and zirconium is present in an amount of 0.015 wt. %.

In a further embodiment, there is provided a process for producing methanol by reacting carbon dioxide with hydrogen over an enhanced catalyst as herein described.

In one embodiment the hydrogen to carbon dioxide ratio is within the range of 3:1 to 10:1, the temperature is within the range of 200 to 300° C., the pressure is within the range of 20 to 100 bar and the gas hourly space velocity (GHSV) is within the range of 2160 to 31200 ml/g·h.

### BRIEF DESCRIPTION OF DRAWINGS

It will be convenient to further describe the present invention with respect to the accompanying drawings that illustrate possible arrangements of the invention. Other arrangements of the invention are possible, and consequently the particularity of the accompanying drawings is not to be understood as superseding the generality of the preceding description of the invention.

FIG. 1 is a schematic overview of the system for CO₂ hydrogenation to methanol according to an embodiment of the invention.

### DETAILED DESCRIPTION

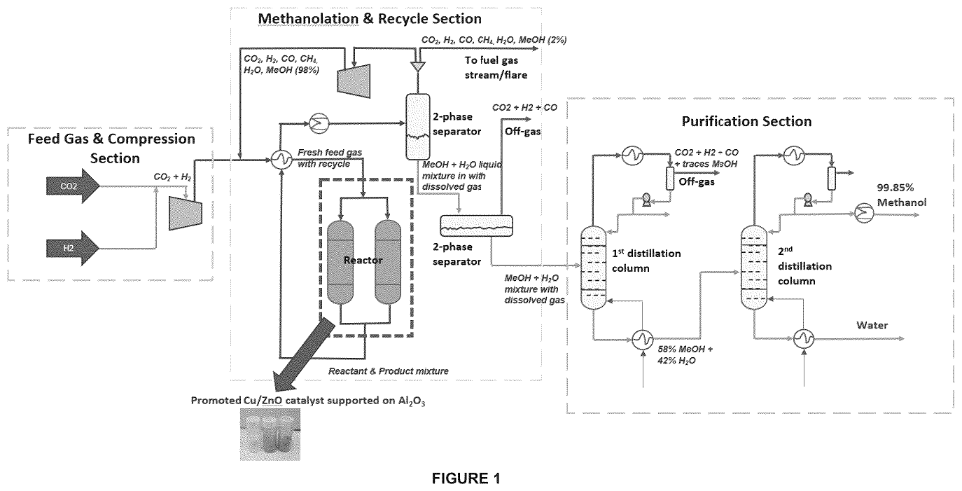

With regard to FIG. 1, a schematic overview of the catalyst utilised in the system for CO₂ hydrogenation to methanol is illustrated.

The present invention describes an enhanced catalyst for carbon dioxide hydrogenation to methanol comprising a copper-zinc oxide catalyst supported on Al₂O₃ promoted with manganese, niobium and zirconium in an amount of up to 1 wt. %.

The promoted catalyst is subjected to an activation process prior to being utilised in the hydrogenation of carbon dioxide to methanol.

The invention further describes a process for producing methanol by reacting carbon dioxide with hydrogen over the promoted catalyst.

Further details are provided below.

### Catalyst Preparation

Preparation of Promoted Cu/ZnO Al₂O₃ Catalyst

Cu/ZnO with fixed metal loading of 15 wt. % at a ratio of 7:3 and 0.09 wt. % of total promoters (Mn/Nb/Zr) was prepared using incipient wetness impregnation method. The amount of each precursor and promoter added was calculated based on catalyst mass prepared over Al₂O₃ support.

The metal precursors, copper nitrate trihydrate (Cu(NO₃)₂·3H₂O) and zinc nitrate hexahydrate (Zn(NO₃)₂·6H₂O), together with the promoters, manganese (II) nitrate tetrahydrate (Mn(NO₃)₂·4H₂O), ammonium niobate (V) oxalate hydrate (C₄H₄NNbO₉·xH₂O) and zirconium (IV) oxynitrate hydrate (ZrO(NO₃)₂·H₂O) were dissolved in deionized water to produce a 0.5M aqueous solution. The solution was stirred using a magnetic stir bar on a hotplate stirrer for one hour. The prepared aqueous precursor solution was then added dropwise using a pipette to a beaker containing Al₂O₃ support in the form of powder.

The pH of the mixture was kept at 7 by employing either a 10% ammonia solution or a 10% nitric acid solution during the addition of the precursor and promoter solution. The mixture was stirred for 24 hours, filtered, and washed with deionized water. The paste formed was dried in an oven at 120° C. for 12 hours. The dried catalyst was then placed in a ceramic crucible and calcined for 4 hours in an air muffle furnace chamber at 350° C.

The alumina support utilised in the present invention results in outstanding catalytic performance with high CO₂ conversion, methanol selectivity and methanol yield. In addition, alumina in comparison to SBA-15 utilised in the prior art is more stable and cost efficient. Further, SBA-15 is not recyclable which result in very poor catalytic performance.

### Promoter Ratio Evaluation

The promoted Cu/ZnO Al₂O₃ catalyst was evaluated using default hydrogenation operating conditions (reduction temperature of 250° C., reaction temperature of 250° C., pressure of 22.5 bar, gas hourly space velocity (GHSV) of 10,800 ml/g·h and H₂:CO₂=3:1) to determine the promoter ratio combination (Mn, Zr and Nb) that resulted in highest methanol yield.

The promoted catalyst formulation that resulted in highest methanol yield was selected for further optimization for activation and hydrogenation process reactions.

### Catalyst Activation

The promoted Cu/ZnO Al₂O₃ catalyst was activated under hydrogen flow within the range of 20-1000 ml/min, at a pressure within the range of 1 to 10 bar, at a reduction temperature within the range of 190-390° C., at a ramping rate within the range of 1 to 38° C./min for a duration of 1 to 7 hours prior to the hydrogenation reaction.

Activation is necessary to reduce the conversion of metal oxide to metallic form so that the catalyst is active during the hydrogenation reaction. The optimised activation conditions ensure the promoted catalyst is fully reduced which allows for outstanding catalytic performance with high CO₂ conversion, methanol selectivity and methanol yield.

### Procedure for CO₂ Hydrogenation Reaction

The calcined promoted catalyst sample was placed into a reactor tube of a fixed bed reactor, sandwiched between layers of quartz wools. The reactor was then purged with He or N₂ to remove impurities and ensure inert conditions. Activation of the catalyst was carried out at identified activation conditions. After the promoted Cu/ZnO Al₂O₃ catalyst was activated, reactant gases (H₂ and CO₂) at identified ratio (3:1-10:1) is fed into a reactor column at a total flow rate of 30-600 ml/min. The reaction temperature is set within the range of 200 to 300° C. with reaction pressure within the range of 20 to 100 bar (pressurized system using He/CO₂ and H₂) and gas hourly space velocity (GHSV) within the range of 2160 to 31200 ml/g·h (which translates from the catalyst mass and total flow rate used). An increase in pressure and H₂:CO₂ ratio further increases methanol selectivity and methanol yield. The hydrogenation reaction was performed for 5 hours up to 30 days for catalyst stability investigation.

Table 1 illustrates catalyst performance data where under comparative conditions, the present invention with a catalyst formulation promoted with Mn, Nb and Zr (in the ratio of 4:1:1 with up to 1 wt % of overall Cu/ZnO weight) has a methanol yield of 63.59% whereas the other catalyst formulations promoted with Mn, Nb and Zr (in the ratio of 1:1:1) supported on either Al₂O₃ or SBA-15 has a methanol yield <19.40%. The increased methanol yield is targeted to reduce the recycling ratio with expected reduction in operating costs.

TABLE 1

| | | | Activation conditions | | | Reaction conditions | | | | Catalytic performance | | |
|---|---|---|---|---|---|---|---|---|---|---|---|---|
| | | | Temper- | Ramping | | | Temper- | | | CO₂ | MeOH | MeOH |
| Support | Active | Promoter's | ature | rate | Duration | | ature | Pressure | GHSV | conversion | selectivity | yield |
| type | precursor | ratio | (° C.) | (° C./min) | (h) | H₂:CO₂ | (° C.) | (bar) | (ml/g · h) | (%) | (%) | (%) |
| Al₂O₃ | Cu/ZnO | Mn:Nb:Zr | 250 | 10 | 2 | 3:1 | 250 | 22.5 | 10800 | 12.47 | 60.23 | 7.50 |
| (powder) | | (1:1:1) | | | | | | | | | | |
| Al₂O₃ | | Mn:Nb:Zr | 250 | 10 | 2 | 3:1 | 300 | 40 | 2160 | 24.04 | 64.95 | 15.61 |
| (pellet) | | (1:1:1) | | | | | | | | | | |
| SBA-15 | | Mn:Nb:Zr | 250 | 10 | 2 | 3:1 | 250 | 22.5 | 10800 | 19.60 | 99.00 | 19.40 |
| (powder) | | (1:1:1) | | | | | | | | | | |
| Al₂O₃ | | Mn:Nb:Zr | 243 | 23 | 2 | 10:1 | 300 | 80 | 6240 | 68.35 | 93.00 | 63.59 |
| (powder) | | (4:1:1) | | | | | | | | | | |

It will be appreciated by persons skilled in the art that the present invention may also include further additional modifications made to the system which does not affect the overall functioning of the system

1. An enhanced catalyst for carbon dioxide hydrogenation to methanol comprising;
a copper-zinc oxide catalyst supported on an alumina support;
and at least one promoter;
wherein the promoter is added to the copper-zinc oxide catalyst in an amount of up to 1 wt. %. to form a promoted copper-zinc oxide catalyst on the alumina support.

2. The enhanced catalyst according to claim 1 wherein the promoted copper-zinc oxide catalyst is activated under a hydrogen flow within the range of 20 to 1000 ml/min, at a pressure within the range of 1 to 10 bar, at a reduction temperature within the range of 190 to 390° C., at a ramping rate within the range of 1 to 38° C./min for a duration of 1 to 7 hours.

3. The enhanced catalyst according to claim 1 wherein the alumina support is aluminium oxide.

4. The enhanced catalyst according to claim 1 wherein the at least one promoter is selected from manganese, niobium and zirconium.

5. The enhanced catalyst according to claim 1 wherein the at least one promoter is a combination of manganese, niobium and zirconium in the ratio of 4:1:1.

6. The enhanced catalyst according to claim 1 wherein manganese is present in an amount of 0.06 wt. %, niobium is present in an amount of 0.015 wt. % and zirconium is present in an amount of 0.015 wt. %.

7. A process for producing methanol by reacting carbon dioxide with hydrogen over an enhanced catalyst according to claim 1.

8. The process according to claim 7 wherein the hydrogen to carbon dioxide ratio is within the range of 3:1 to 10:1, the temperature is within the range of 200 to 300° C., the pressure is within the range of 20 to 100 bar and the gas hourly space velocity (GHSV) is within the range of 2160 to 31200 ml/g·h.
